(19) United States
(12) Patent Application Publication
(10) Pub. No.: US 2013/0018329 A1
(43) Pub. Date: Jan. 17, 2013

(54) PORTABLE AMPOULE WITH A SPECIALIZED TIP AND SEALER

(71) Applicant: Ketan C. Mehta, Santa Rosa, CA (US)

(72) Inventor: Ketan C. Mehta, Santa Rosa, CA (US)

(21) Appl. No.: 13/183,387

(22) Filed: Jul. 14, 2011

(51) Int. Cl. A61M 3/02

(52) U.S. Cl. 604/246

(57) ABSTRACT

The present disclosure relates to a portable ampoule with a specialized tip and sealer. In a general aspect, the portable ampoule for dispensing fluid may include a body configured to contain cleansing solution. A neck may be coupled to the body and configured to control the flow of the solution. A tip may be coupled to the neck and have an aperture for solution release. A sealing device may be coupled to the tip and configured to seal the aperture. The sealing device may permanently unseal the aperture upon decoupling from the tip.

### TECHNICAL FIELD

The subject matter of this application is generally related to dispensers.

### BACKGROUND

People in many parts of the world perform nasal cleansing (or nasal irrigation) using a neti pots or other products on a routine basis. Nasal cleansing is also incorporated into some forms of yoga practice, such as in Jala neti. Jala neti is a Sanskrit term that refers to cleansing and means “water cleansing”. The solution for rinsing the nasal passages can be a saline solution. Some patients use nasal rinsing to reduce allergies, improve breathing, eliminate post-nasal drip or sinus infections, moisten dry nasal passages, avoid catching a cold, or otherwise generally improve one's health. Other uses are possible. Conventional nasal rinse products, however, are bulky and do not fit within purses, backpacks, briefcases, or other personal items that are carried around.

### SUMMARY

The present disclosure relates to a portable ampoule with a specialized tip and sealer. In a general aspect, the portable ampoule for dispensing fluid may include a body configured to contain cleansing solution. A neck may be coupled to the body and configured to control the flow of the solution. A tip may be coupled to the neck and have an aperture for solution release. A sealing device may be coupled to the tip and configured to seal the aperture. The sealing device may permanently unseal the aperture upon decoupling from the tip.

Implementations may include one or more of the following features. The sealing device may be for a single use such that upon removing the sealing device from the tip, the sealing device is permanently displaced from the tip. The sealing device may include an opener having a twist coupler to facilitate twisting motion for removing the sealing device to unseal the aperture. The opener may be a planar holding structure to accommodate application of moment or torque to the twist coupler. The opener may include an outer rim to provide torque support and an inner rim to secure the twist coupler to the outer rim. The sealing device may include a sealer and the twist coupler may be coupled to the tip through the sealer.

The body may include a rib structure to facilitate holding or gripping of the body to avoid slipping motion generated when the body is rotated in an opposite direction with respect to the sealing device. The rib structure may be flush or contiguous with edges of the opener. The rib structure may extend from a bottom portion of the tip to a bottom portion of the body, wherein a bottom surface of the opener body aligns with the bottom portion of the tip. The twist coupler includes inner side walls that conform with but do not contact sidewalls of the tip.

The tip may be further configured to attenuate the pressure of solution stored in the body and facilitate dispensing of the solution with sufficient pressure to deliver the solution to nasal tissue without displacing the nasal tissue. The neck may have a diameter smaller than a diameter of the body to facilitate a dispensing speed of the solution. The tip may be conically shaped with a convex curved surface tapered from a surface on which the aperture is formed to a bottom portion of the tip. The bottom portion may include a diameter with sufficient dimension to prevent the tip from extending into a user's nostril. The bottom portion of the tip may include rounded or chamfered edges. The tip may include a tapered surface that conforms to nostrils of different sizes.

The details of one or more implementations of the subject matter described in this specification are set forth in the accompanying drawings and the description below. Other features, aspects, and advantages of the subject matter will become apparent from the description, the drawings, and the claims

### BRIEF DESCRIPTION OF DRAWINGS

FIG. 1A is a schematic front view of an ampoule.

FIG. 1B is a schematic top view of the ampoule.

FIG. 2 is a schematic side view of the ampoule.

FIG. 3 is a schematic cross-sectional view at A-A of the ampoule.

FIGS. 4A and 4B are schematic views of the ampoule before and after opening respectively.

FIG. 5A is a schematic view of a second ampoule implementation.

FIG. 5B is a schematic cross-sectional view along the longitudinal axis of the second ampoule implementation.

Like reference symbols in the various drawings indicate like elements.

### DETAILED DESCRIPTION

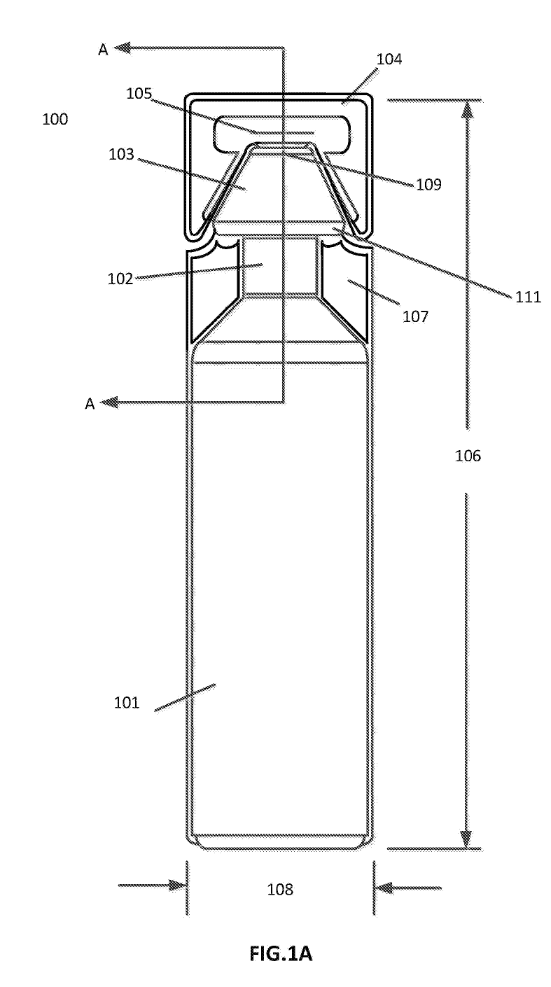

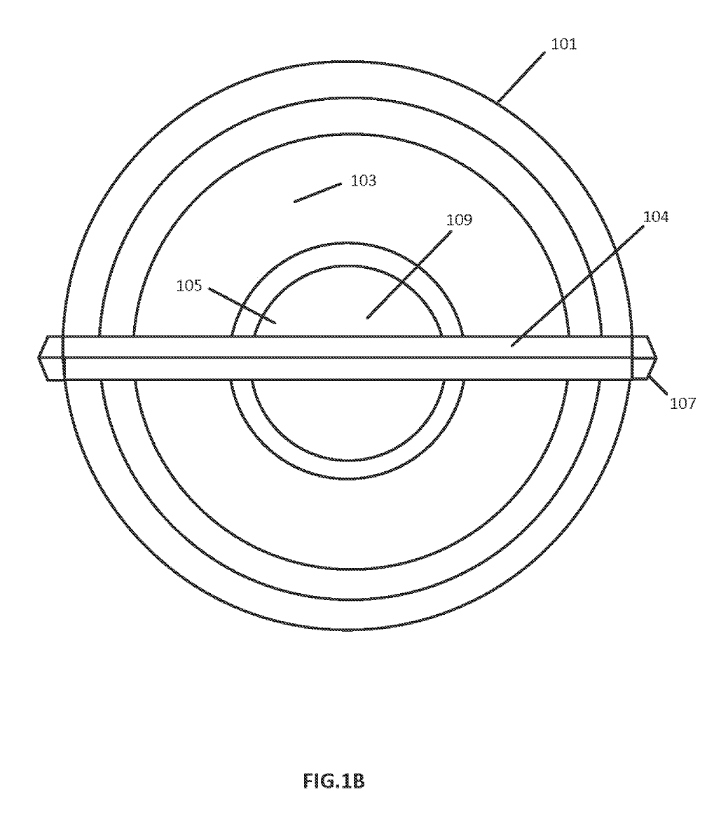

FIG. 1A is a schematic front view of an ampoule 100. As shown in FIG. 1A, the ampoule 100 includes a body 101, a neck 102, a tip 103 and an opener 104. The neck 102 connects the body 101 to the tip 103. The opener 104 allows users to twist open the tip 103 at a twist coupler 105. The body 101 can be, for example, a container of saline solution or any other fluid suitable for irrigating cavities (e.g. nasal cavities). The ampoule 100 can be used, for example, to provide nasal rinsing (or irrigation or nasal lavage), such as to treat allergies, improve breathing, eliminate post-nasal drip or sinus infections, moisten dry nasal passages, etc. In some implementations, the tip 103 can attenuate the pressure of fluid stored in the body 101, dispensing fluid at a gentle pressure. The gentle pressure can be sufficient to deliver a flow of fluid to tissue without the pressure being so great as to displace the tissue.

In some implementations, the body 101 can be a fluid container (e.g. a bottle, can or other container) that securely stores fluid and allows users to apply pressure (e.g. to the container) to expel the stored fluid. For example, the body 101 can be made of thermoplastic polymers, thermosetting polymers, or any other appropriate materials that allows for deformation in order to pressurize the bottle for fluid release. In some implementations, the ampoule 100 can be pressurized for maintaining shape during transportation. In some implementations, the body 101 is a cylindrical shape of a uniform diameter. In some implementations, the diameter can vary along its longitudinal axis, for example, a tapered shape, a curved shape, a diamond shape, or other shapes. The body 101 can be a thin-walled structure of uniform thickness and/or variable thickness for functional requirements. For example, to facilitate deformation, some locations on the body 101 can be thinner than the rest. As another example, other locations on the body 101 can be thicker for structural reinforcement, such as a portion at or near the bottom of the ampoule 100. Grooves or depressions can be included in the body to facilitate gripping by the human hand.

In some implementations, the dimension of the neck 102 can be tailored to accommodate an increase output flow velocity of fluid from the body 101. The neck 102 can be made of the same material as the body 101, such as thermoplastic polymers, thermosetting polymers, etc. In some implementations, the neck 102 is a cylindrical shape of a uniform diameter that is significantly smaller than that of the body 101 (e.g., the diameter of neck 102 is 50% smaller than the maximum diameter of the body 101). Other ratios between the diameter of the neck 102 and that of the body 101 are also contemplated. In some implementations, a small neck diameter allows the output flow velocity to increase (e.g., because for a given amount of fluid volume that is displaced, the narrower the cross-sectional area of a passage, the higher the flow velocity). The neck 102 can also be a thin-walled structure of uniform thickness and/or variable thickness for functional requirements. For example, at the location where the body 101 and the tip 103 intersects, extra wall thickness can be implemented to enhance structural integrity.

As shown in FIG. 1, the tip 103 is connected to the neck 102, which is partially encapsulated by the twist coupler 105. An aperture 109 is revealed upon removing the twist coupler 105. In some implementations, the tip 103 can be conically shaped with a convex curved surface leading from the aperture 109 toward the bottom portion 111 of the tip 103. In some implementations, the tip 103 can be gumdrop- or mushroom-shaped. The tip 103 may include rounded edges such as an upper rounded edge and a lower rounded edge. The rounded edges may have a substantially large rounding radius to facilitate manufacturing process and avoid causing discomfort to user during use and/or handling. For example, the upper rounded edge can be of a rounding radius between 0.5 to 4 mm, such as 1 mm. This allows the tip 103 to comfortably contact with the user at various insertion angles without excessive friction to cause irritation or discomfort. The lower rounded edge may have a rounding radius between 1 to 6 mm, such as 2 mm. This allows the tip 103 to be safely completely enclosed by a user's nostril and cause minimum friction and discomfort during removal. This also allows the tip 103 to avoid stress concentration during production, transportation and use.

The tip 103 can include a tapered surface that permits the tip 103 to conform to nostrils of different sizes. Specifically, the exterior of the tip 103 can be tapered outwardly. For example, the tip 103 tapers from a wide portion up (e.g., the portion near the bottom of the tip 103) to a narrow portion (e.g., the portion near the top of the tip 103). The tip 103 can be sized to prevent the wide portion from extending all the way into a user's nostril. In some implementations, the transition from the end of the wide portion to the neck 102 can be rounded or chamfered to avoid any sharp edges. The tip 103 can be made of the same material as the body 101, such as thermoplastic polymers, thermosetting polymers, or other suitable materials tailored for human use. The tip 103 can be a thin-walled structure of approximate uniform thickness.

In some implementations, the twist coupler 105 is breakably coupled to the tip 103 at the aperture 109 and reinforceably affixed to the opener 104. The opener 104 can be sized to facilitate the twisting motion for twist opening the twist coupler 105. In some implementations, the twist coupler 105 acts as a one-time seal to the tip 103 so that upon twist opening the twist coupler 105, the twist coupler 105 is permanently displaced from the tip 103. In so doing, the tip 103 cannot re-seal the aperture 109, and the ampoule 100 can be discarded after one-time use.

In some implementations, the twist coupler 105 is a thin-walled structure coupled to the tip 103 by, for example, heated compression or any similar techniques, sealingly adhering two adjacent walls that can be broken with a twisting motion when the shear stress exceeds the bonding strength between the two thin walls. The twist coupler 105 can be of a donut shape, a tire shape, or any other appropriate shape to encapsulate the aperture 109. The twist coupler 105 can be made of the same material as the tip 103, such as thermoplastic polymers, thermosetting polymers, and other suitable materials.

In some implementations, the opener 104 is integrally and reinforceably affixed to the twist coupler 105. The opener 104 serves as a holding structure for user's fingers to apply a moment/torque to the twist coupler 105. In some implementations, the opener 104 is a plane structure of a thickness that defies significant bending deformation under normal use. The opener 104 may include an outer rim to provide torque support and an inner rim to secure the twist coupler to the outer rim. For example, the outer rim may be of a thicker thickness than the inner rim so that when a torque is applied to the outer rim, structural deformation is limited by the material strength of the outer rim. The thickness of the outer rim may be between about 1 and 4 mm, such as 2.5 mm. The primary function of the inner rim is to secure the twist coupler 105 to the outer rim. As the inner rim deforms under loading, tensile stress can become the major stress within the component to provide a transmitting force to rotate the twist coupler 105. Therefore, the thickness of the inner rim may not require a large thickness, between about 0.5 to 2 mm, such as 1 mm. This also saves production material and reduces portable weight of the ampoule 100.

In some implementations, the opener 104 is affixed to a cap (not shown) instead of the twist coupler 105 for re-usable purposes. The cap may be a screw type cap that has spiral rails to fasten with the ampoule 100. The aperture 109 may have an intruding structure coupling with the cap. The material for the cap may be flexible to allow deformation to occur to form a liquid-tight fit. This alternation allows user to protect the ampoule 100 before use (e.g., during transportation).

In some implementations, a rib structure 107 is included along the longitudinal axis of the body 101 and in the plane defined by the opener 104. The rib structure 107 allows users to conveniently hold and grip the body 101 and avoid a slipping motion in the rotational direction. For example, a user can use three fingers (e.g. a middle finger, a ring finger and a pinky) to grip around the cylindrical portion of the body 101 and the other two figures (e.g. a thumb and an index finger) to hold the planar portion of the rib structure 107. This finger hold securely restricts motion of the tip 103 (e.g., to restrict the neck 102 and the body 101 from compliant motions such as rotation along with the opener 104). The user can then use the other hand's two fingers (e.g. a thumb and an index finger) to hold the opener 104 by the planar surface (e.g. pressing onto the surface, or to act on the rib portion), and apply a torque/moment to twist the opener 104 against the tip 103. Excessive deformation occurs when the torque exceeds a predetermined value so that the deformation can cause the twist coupler 105 to break away from the tip 103, revealing the aperture 109. Therefore, the body 101 includes an outer rim (i.e. the planar portion of the rib structure 107 and the opener 104) to provide torque support and an inner rim (i.e. the material between the tip 103 and the opener 104) to secure the twist coupler 105 to the outer rim.

As shown in FIG. 1, the opener 104 and the rib structure 1007 are separated below the bottom of the tip 103. This allows the tip 103 be completely inserted into a user's nostril without obstruction and/or causing discomfort. The rib structure 107 may be confined to a contour that avoids contact with a user's nostril when the tip 103 is fully inserted. Although the opener 104 and the rib structure 107 are shown separated near the bottom of the tip 103, other implementations also are contemplated in which the separation gap is placed at different locations, such as the shoulder of the body 101 or anywhere between the shoulder of the body 101 and the twist coupler 105. These examples, however, are non-limiting. Also, the opener 104 can have two steps of thicknesses: an outer rim for major torque support and an inner portion for securing the twist coupler 105 to the outer rim. In some implementations, the inner portion of the opener 104 can be of the same thickness as the rib structure 107, and does not contact the sidewalls of the tip 103. In the implementation illustrated in FIG. 1, the edges of the opener 104 are flush with the bottom of the tip 103 to facilitate the opening operation. The edges of the opener 104 is also flush or contiguous with the rib structure 107 of the body 101.

The bottom of the body 101 can include a dimension 108. The dimension 108 can include the diameter of the body 101 and a side extrusion step from the rib structure 107. To enhance portability and miniaturize the ampoule 100, the dimension 108 can be between about 15 mm and 25 mm (e.g., about 22 mm), and the extrusion portion of the rib structure 107 can be, for example, 1.5 mm. The diameter of the body 101 can be of any other values that, given certain length, can contain enough fluid for a one-time treatment, such as rinse, lavage, moisturize, etc. Various dimensions of the ampoule 100 also can exist. For example, the overall length 106 of the ampoule 100 can be between about 80 mm and 120 mm (e.g., 99 mm). The overall length 106 can be of any other value that fits within conventional purses, backpacks, briefcases, or other daily carry items.

FIG. 1B is a schematic top view of the ampoule 100, as shown in FIG. 1A. In some implementations, the body 101, the tip 103, and the twist coupler 105 have circular cross section shape at various diameters. For example, the cross section of the twist coupler 105 may be a circular shape that has a diameter between about 4 and 10 mm (e.g., 6.35 mm). The maximum cross section of the tip 103 may have a diameter between about 10 and 20 mm (e.g., 15 mm). The cross section of the body 101 may have a diameter between about 15 and 25 mm (e.g., 22 mm) as the dimension 108. It can be seen from the top view that the opener 104 and the rib structure 107 align in the same plane that symmetrically divide the ampoule 100. Although the general cross section of the ampoule 100 is circular shape in this example, the cross section may be, in some implementations, a different practical shape, such as an elliptical shape for ease of applying pressure, a triangular shape for packaging reasons, a diamond shape for both ease of applying pressure and packaging reasons, and/or a combination of different shapes at different cross section locations.

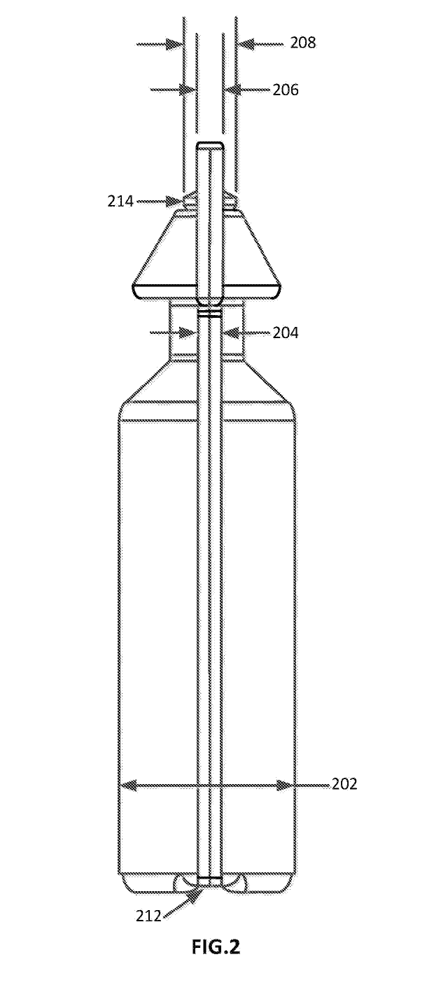

FIG. 2 is a schematic side view of the ampoule 100, as shown in FIG. 1A. The side view shows additional structures of the ampoule 100. For example, the bottom of the body 101 is shaped for reinforcement and easy mold release that includes a concave surface 212 in the extrusion direction of the rib structure 107. Also, as shown in FIG. 2, the twist coupler 105 is attached to the tip 103 at a circular tangential portion 214 that acts as a plug or sealer for sealing the aperture 109. Further, in the example shown, the outer portion of the opener 104 is thicker than the rib structure 107. FIG. 2 further shows a dimension 202 to denote the diameter of the body 101, a dimension 204 to denote the thickness of the rib structure 107, a dimension 206 to denote the thickness of the opener 104, and a dimension 208 to denote the diameter of the twist coupler 105.

The circular tangential portion 214 connects the twist coupler 105 to the sealing aperture 109 by a cross section that has sufficient strength to maintain structural integrity during transportation (i.e. maintain shape under bending and tension loading conditions) and can be severed under shear stress in a twisting motion. In the example shown in FIG. 2, the circular tangential portion 214 has a low aspect ratio (e.g., height to diameter ratio is very small), allowing for very small moment arm for bending deformation. This shape profile enables resistance against bending failure modes. The circular tangential portion 214 is affixed to the opener 104 that allows for a large moment arm to be applied by user (at least about twice as large as the sealing cross section diameter). This allows for more material to be in contact at the sealing cross section between the circular tangential portion 214 and the aperture 109, better resisting tension or compression deformation.

To expose the aperture 109, a moment is applied to the opener 104 that is affixed to the twist coupler 105 by circumferential connection. The moment creates a shear stress concentrated at the circular tangential portion 214 while the connection between the twist coupler 105 and the opener 104 is under tension. Such as tearing apart a piece of paper is much easier than pulling apart a piece of paper, the cross section between the aperture 109 and the circular tangential portion 214 will fail or break before any other locations. This breaks apart the opener 104 and the tip 103 and exposes the aperture 109. The twist coupler 105 may include inner side walls that conform with but do not contact sidewalls of the tip 103.

In some implementations, the circular tangential portion 214 may have a donut-shape, a tire shape, or any other low aspect ratio cylindrical shapes that enable separation from the aperture 109 with shear stress. In some implementations, the circular tangential portion 214 may be of the same cross section shape as the aperture 109 and/or the tip 103.

The concave surface 212 at the bottom of the body 101 illustrated in FIG. 2 has multiple purposes, such as reinforcing the structural integrity of the body 101, enabling faster manufacturing process, allowing user to recognize the ampoule orientation, etc. In some implementations, the concave surface 212 creates a strengthening profile of the bottom of the body 101 by increasing the moment of inertia of the structure. This is the similar principle applied to most thin-walled bottles that use the shape instead of materials to achieve certain desired strength. The concave surface 212 also creates a strong local structure of the body 101 to withstand relatively large external loads. This may facilitate the manufacturing process when the body 101 is to be handled by various machines.

Various dimensions of the ampoule 100 are possible and illustrated in FIG. 2. For example, the diameter 202 of the body 101 can be in the range between 12 and 30 mm (e.g., about 20.5 mm). The diameter 202 can be of any other values that, given certain length, can contain enough fluid for a one-time treatment. In some implementations, the thickness 204 of the rib structure 107 can be in the range of 1 to 2 mm (e.g., about 1.4 mm). In some implementations, the thickness 204 can be of any other values so that, when loaded to twist open the ampoule 100, the rib structure 107 can maintain the original shape without excessive deformation.

In some implementations, the thickness 206 of the opener 104 can be in the range between 2 and 3 mm (e.g., about 2.4 mm). In some implementations, the thickness 206 can be at least 1 mm thicker than the thickness 204, or of any other values that gives the opener 204 enough structure integrity to twist open the coupler 105. In some implementations, the diameter 208 of the circular tangential portion 214 can be in the range between 3 and 8 mm (e.g., about 6.35 mm). The diameter 208 can be of any other values sufficient to provide a secure seal to the aperture 109.

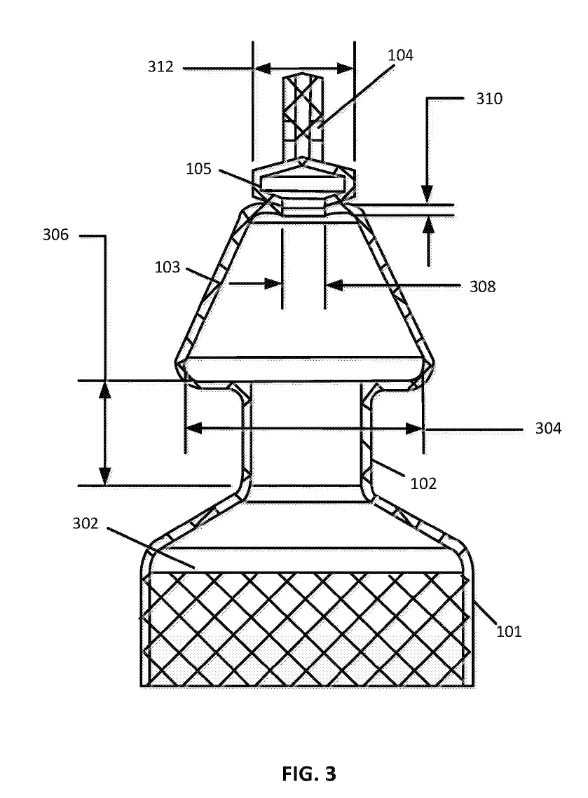

FIG. 3 is a schematic cross-sectional view of the ampoule 100. As shown in FIG. 3, portion of the body 101 is shown with a line 302 indicating the fill-up line for the fluid contained in the body 101. At about 20 ml fluid volume and about 20 mm body diameter, the line 302 can be about 66 mm from the bottom of the body 101. The position of the line 302 can change if a different fluid volume is to be filled and the body 101 is of a different diameter or size. The length 306 of the neck 102 can be in the range between 2 and 8 mm (e.g., about 6.4 mm), and can be of any other values that provides the rib structure 107 enough room for holding the ampoule 100.

In some implementations the rib structure 107 at the body 101 can be flush or contiguous with the rib structure 107 at the opener 104. In some implementations, the bottom diameter 304 of the tip 103 can be in the range between 10 and 20 mm (e.g., 14.9 mm), while the top diameter 312 of the tip 103 can be in the range between 3 and 8 mm (e.g., 6.35 mm), or the same value as the diameter 208. In some implementations, the diameter 308 of the aperture 109 can be in the range between 1.5 and 3.5 mm (e.g., 2.54 mm). In some implementations, the overall structure can be of a uniform thickness 310, which can be in the range between 0.3 and 0.8 mm (e.g., 0.65 mm).

As discussed above, the ampoule 100 can be made of a thermoplastic polymer, or thermoplastics. Most thermoplastics are high-molecular-weight polymers whose chains associate through weak Van der Waals forces (e.g. polyethylene); stronger dipole-dipole interactions and hydrogen bonding (e.g. nylon); or even stacking of aromatic rings (e.g. polystyrene). For example, the ampoule 100 can be made of acrylonitrile butadiene styrene, acrylic, celluloid, cellulose acetate, cyclic olefin copolymer, ethylene-vinyl acetate, ethylene vinyl alcohol, fluoroplastics, lonomers, Kydex, liquid crystal polymer, polyoxymethylyne, polyacrylates, polyacrylonitrile, polyamide, polyamide-imide, polyaryletherketone, polybutadiene, polybutylene, polybutylene terephthalate, polycaprolactone, polychlorotrifluoroethylene, polyethylene terephthalate, polycyclohexylene dimethylene terephthalate, polycarbonate, polyhydroxyalkanoates, polyketone, polyester, polyethylene, polyetheretherketone, polyetherketoneketone, polyetherimide, polyethersulfone, chlorinated polyethylene, polyimide, polylactic acid, polymethylpentene, polyphenylene oxide, polyphenylene sulfide, polyphthalamide, polypropylene, polystyrene, polysulfone, polytrimethylene terephthalate, polyurethane, polyvinyl acetate, polyvinyl chloride, polyvinylidene chloride, styrene-acrylonitrile, and/or a combination of these, or any other appropriate thermoplastics.

In some implementations, the ampoule 100 can be made of a thermosetting polymer, or thermoset. Thermoset is a polymer material that cures irreversibly through heat (generally above 200° C. (392° F.)), through a chemical reaction (two-part epoxy, for example), or irradiation such as electron beam processing. In some instances, the ampoule 100 can be made of vulcanized rubber, bakelite, duroplast, melamine resin, phenol formaldehyde, urea formaldehyde, melamine formaldehyde, polyester, epoxy, isoprene crosslinked with sulphur, neoprene, trihydroxymethylsilane, and/or a combination of these, or any other appropriate thermosetting polymers.

In some implementations, the ampoule 100 can be coated internally with a layer of epoxy resin to prevent reaction between the fluid and the body material. For example, if the ampoule 100 is coated with a layer of metal for light isolation or uses a metallic material for construction, then a layer of epoxy resin can provide isolation of the fluid and prevent undesired materials leaching into the liquid or solution contained in the body 101.

In some implementations, the ampoule 100 can be transparent overall, or in a portion such that remaining portion of fluid may be monitored. For example, the ampoule 100 may be made of a clear thermoplastic polymer, and/or a clear thermosetting polymer.

In some implementations, the ampoule 100 can use various materials for the body 101, the neck 102 and the tip 103. For example, the body 101 can use a thermoplastic polymer while the neck 102 and the tip 103 can use a thermosetting polymer. A variation of material in different parts of the ampoule 100 can improve durability, provide convenience to use, or enhance other characteristics of the ampoule 100 such as gripping.

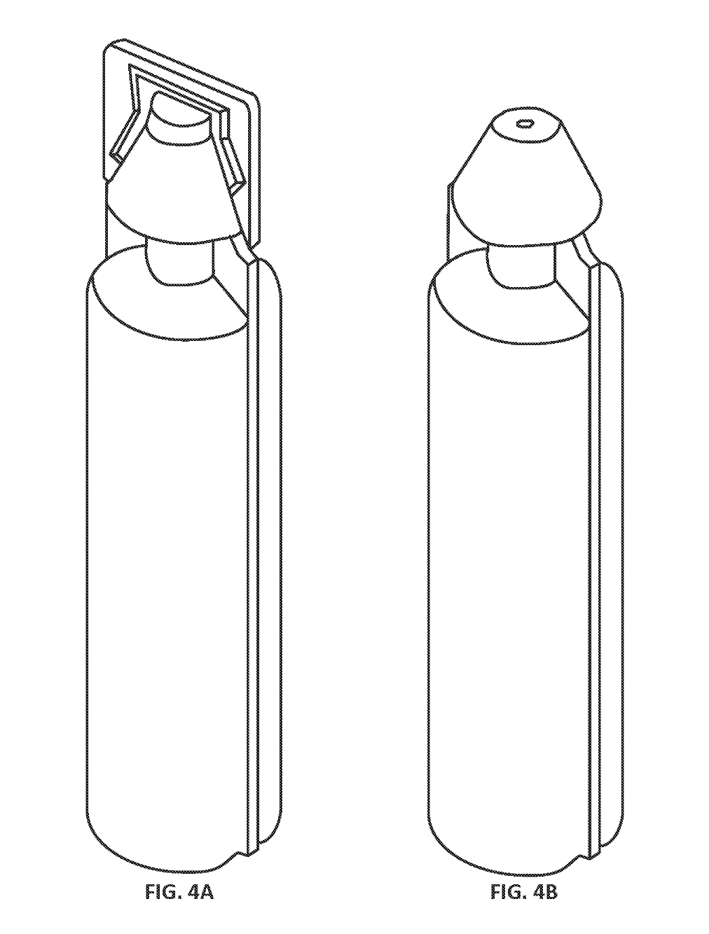

FIG. 4A is a one schematic view of the ampoule 100 before removing the opener 104, and FIG. 4B is another schematic view of the ampoule 100 after removing the opener 104. As shown in FIG. 4A, the opener 104 and the twist coupler 105 are attached to the tip 103. The rib structure 107 allows users to conveniently hold and grip the body 101 and avoid slipping motion in the rotational direction. Upon removing the opener 104, the aperture 109 is revealed.

In some implementations, a user should be in a upright position before using the opened ampoule 100, which is shown in FIG. 4B. The head of the user can be tilted to one side slightly. After placing the tip 103 into one nostril, the user can press gently to dispense a few drops or a small quantity for moisture or squeeze to expel a larger quantity for nasal irrigation. After one use, the whole ampoule 100 can be discarded, along with unused solution. The ampoule 100 can be used to contain fluid that is a drug-free, preservative-free, sterile nasal saline solution. The solution can sooth and moisturize dry and congested noses for babies, children and adults. In the 20 ml volume implementation shown in FIG. 3, the ampoule 100 is convenient for home, nursery, playground, school, air travel, hotel room and hospital use. A few drops of saline can moisturize, while squeezing a larger quantity can deliver a gentle low pressure low volume nasal rinse directly into the nostril for stronger results.

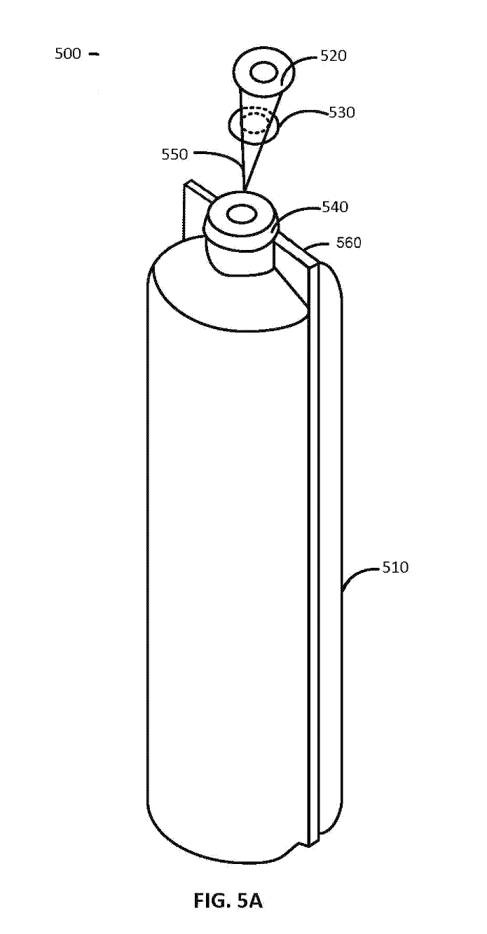

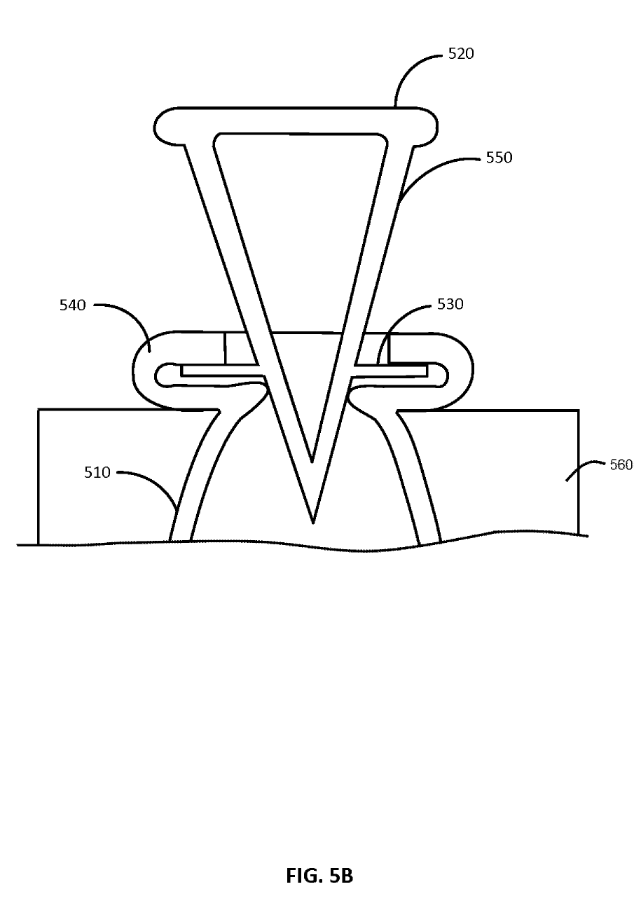

FIG. 5A is a schematic view of a second ampoule implementation 500. In this implementation, the second ampoule 500 includes a body 510 and a cap 550. The body 510 can be a fluid container (e.g. a bottle, can or other container) that securely stores fluid and allows users to apply pressure (e.g. to the container) to expel the stored fluid. For example, the body 510 can be made of thermoplastic polymers, thermosetting polymers, or any other appropriate materials that allows for deformation in order to pressurize the bottle for fluid release. In some implementations, the ampoule 500 can be pressurized for maintaining shape during transportation. In some implementations, the body 510 is a cylindrical shape of a uniform diameter. In some implementations, the diameter can vary along its longitudinal axis, for example, a tapered shape, a curved shape, a diamond shape, or other shapes. The body 510 can be a thin-walled structure of uniform thickness and/or variable thickness for functional requirements. For example, to facilitate deformation, some locations on the body 510 can be thinner than the rest. As another example, other locations on the body 510 can be thicker for structural reinforcement, such as a portion at or near the bottom of the ampoule 500. Grooves or depressions can be included in the body to facilitate gripping by the human hand.

The body 510 may include a rib structure 560 to facilitate holding or gripping of the body 510 to avoid slipping motion. The rib structure 560 may be flush or contiguous with edges of the opener 520, or may be only extended to a functional portion around the body 510. The rib structure 560 may extend from a bottom portion of the tip to a bottom portion of the body 510.

As shown in FIG. 5A, the cap 550 is a conical needle head structure for securely sealing the ampoule body 510 and allowing for reuse. The cap 550 includes two structural features besides the needle head shape: a pulling support 520 and a sealing support 530. The pulling support 520 enables user to apply a tension force to separate the cap 550 from the body 510. The pulling support 520 may be a sudden increase in diameter of the conical shape of the cap 550. This resulting step structure allows user's fingers to engage with the cap 550. The sealing support 530 is an extruding structure near the middle location of the cap 550. The sealing support 530 engages with the sealing end 540 at the tip of the body 510. The sealing end 540 may be a donut, a tire or other inner grooved shape that couples with the sealing support 530 under a predetermined stress that seals the aperture of the body 510.

In some implementations, the ampoule 500 can be made of a thermoplastic polymer, or thermoplastics. Most thermoplastics are high-molecular-weight polymers whose chains associate through weak Van der Waals forces (e.g. polyethylene); stronger dipole-dipole interactions and hydrogen bonding (e.g. nylon); or even stacking of aromatic rings (e.g. polystyrene). For example, the ampoule 500 can be made of acrylonitrile butadiene styrene, acrylic, celluloid, cellulose acetate, cyclic olefin copolymer, ethylene-vinyl acetate, ethylene vinyl alcohol, fluoroplastics, lonomers, Kydex, liquid crystal polymer, polyoxymethylyne, polyacrylates, polyacrylonitrile, polyamide, polyamide-imide, polyaryletherketone, polybutadiene, polybutylene, polybutylene terephthalate, polycaprolactone, polychlorotrifluoroethylene, polyethylene terephthalate, polycyclohexylene dimethylene terephthalate, polycarbonate, polyhydroxyalkanoates, polyketone, polyester, polyethylene, polyetheretherketone, polyetherketoneketone, polyetherimide, polyethersulfone, chlorinated polyethylene, polyimide, polylactic acid, polymethylpentene, polyphenylene oxide, polyphenylene sulfide, polyphthalamide, polypropylene, polystyrene, polysulfone, polytrimethylene terephthalate, polyurethane, polyvinyl acetate, polyvinyl chloride, polyvinylidene chloride, styrene-acrylonitrile, and/or a combination of these, or any other appropriate thermoplastics.

In some implementations, the ampoule 500 can be made of a thermosetting polymer, or thermoset. Thermoset is a polymer material that cures irreversibly through heat (generally above 200° C. (392° F.)), through a chemical reaction (two-part epoxy, for example), or irradiation such as electron beam processing. In some instances, the ampoule 500 can be made of vulcanized rubber, bakelite, duroplast, melamine resin, phenol formaldehyde, urea formaldehyde, melamine formaldehyde, polyester, epoxy, isoprene crosslinked with sulphur, neoprene, trihydroxymethylsilane, and/or a combination of these, or any other appropriate thermosetting polymers.

In some implementations, the ampoule 500 can be coated internally with a layer of epoxy resin to prevent reaction between the fluid and the body material. For example, if the ampoule 500 is coated with a layer of metal for light isolation or uses a metallic material for construction, then a layer of epoxy resin can provide isolation of the fluid and prevent undesired materials leaching into the liquid or solution contained in the body 510.

In some implementations, the ampoule 500 can be transparent overall, or in a portion such that remaining portion of fluid may be monitored. For example, the ampoule 500 may be made of a clear thermoplastic polymer, and/or a clear thermosetting polymer.

In some implementations, the sealing support 530 may be made of a material different from that of the body 510, such as metal, for preferred elastic modulus and reliability. The sealing support 530 may be made in a shape that conforms to the inner chamber of the sealing end 540. The shape of the sealing support 530 may experience substantial elastic deformation during the coupling and/or decoupling process with the sealing end 540. In some cases, the sealing support 530 may be made of a foil of stainless steel forming a donut shape to couple with the sealing end 540.

FIG. 5B is a schematic cross-sectional view along the longitudinal axis of the second ampoule implementation. This cross-sectional view shows details about the engagement between the cap 550 and the body 510. The sealing end 540 at the tip of the body 510 may have two inner edges that conform to the tapered needle cap 550. The upper edge of the sealing end 540 may provide a sealing force closing the cap 550 towards the lower edge of the sealing end 540. The cap 550 can be a thin-walled structure of approximate uniform thickness. The cap 550 may experience substantial elastic deformation during the coupling and/or decoupling process with the body 510 at the sealing end 540.

A number of embodiments of the disclosure have been described. Nevertheless, it will be understood that various modifications can be made without departing from the spirit and scope of the disclosure. For example, instead of attenuating a fast stream of liquid into a gentle flow, a mist exiting the actuator can be transformed into a gentle cleansing stream of fluid. Accordingly, other embodiments are within the scope of the following claims.

What is claimed is:

1. A dispensing device comprising:
a body configured to contain solution;
a neck coupled to the body and configured to control the flow of the solution;
a tip coupled to the neck, the tip having an aperture thereon through which the solution is released; and
a sealing device coupled to the tip and configured to seal the aperture, where the sealing device permanently unseals the aperture upon decoupling the sealing device from the tip.

2. The dispensing device of claim 1, wherein the sealing device is for a single use such that upon removing the sealing device from the tip, the sealing device is permanently displaced from the tip.

3. The dispensing device of claim 1, wherein the sealing device includes an opener having a twist coupler to facilitate twisting motion for removing the sealing device to unseal the aperture.

4. The dispensing device of claim 3, wherein the opener is a planar holding structure to accommodate application of moment or torque to the twist coupler.

5. The dispensing device of claim 3, wherein the opener includes an outer rim to provide torque support and an inner rim to secure the twist coupler to the outer rim.

6. The dispensing device of claim 3, wherein the sealing device includes a sealer and the twist coupler is coupled to the tip 103 through the sealer.

7. The dispensing device of claim 3, wherein the body includes a rib structure to facilitate holding or gripping of the body to avoid a slipping motion generated when the body is rotated in an opposite direction with respect to the sealing device.

8. The dispensing device of claim 7, wherein the rib structure is flush or contiguous with edges of the opener.

9. The dispensing device of claim 7, wherein the rib structure extends from a bottom portion of the tip to a bottom portion of the body.

10. The dispensing device of claim 9, wherein a bottom surface of the opener aligns with the bottom portion of the tip.

11. The dispensing device of claim 3, wherein the twist coupler includes inner sidewalls that conform with but do not contact sidewalls of the tip.

12. The dispensing device of claim 1, wherein the tip is further configured to attenuate a pressure of solution stored in the body and facilitate dispensing of the solution with sufficient pressure to deliver the solution to tissue without displacing the tissue.

13. The dispensing device of claim 1, wherein the neck has a diameter smaller than a diameter of the body to facilitate a dispensing speed of the solution.

14. The dispensing device of claim 1, wherein the tip is conically shaped with a convex curved surface tapered from a surface on which the aperture is formed to a bottom portion of the tip.

15. The dispensing device of claim 14, wherein the bottom portion includes a diameter with sufficient dimension to prevent the tip from extending into a user's nostril.

16. The dispensing device of claim 14, wherein the bottom portion of the tip includes rounded or chamfered edges.

17. The dispensing device of claim 1, wherein the tip includes a tapered surface that conforms to nostrils of different sizes.

18. The dispensing device of claim 1, further comprising a removable cap having a conical needle head structure for securely re-sealing the aperture to allow for reuse.

19. The dispensing device of claim 1, wherein the removable cap includes a pulling structure to facilitate the removal of the removable cap and a sealing structure to facilitate sealing of the aperture.
